(19) United States
(12) Patent Application Publication
(10) Pub. No.: US 2025/0228526 A1
(43) Pub. Date: Jul. 17, 2025

(54) ULTRASOUND TRANSDUCER AND MEDICAL DEVICE

(71) Applicant: OLYMPUS MEDICAL SYSTEMS CORP., Tokyo (JP)

(72) Inventor: Satoshi YOSHIDA, Kawagoe-shi (JP)

(73) Assignee: OLYMPUS MEDICAL SYSTEMS CORP., Tokyo (JP)

(21) Appl. No.: 19/171,785

(22) Filed: Apr. 7, 2025

Related U.S. Application Data
(63) Continuation of application No. PCT/JP2022/038484, filed on Oct. 14, 2022.

(51) Int. Cl. A61B 8/00; A61B 8/12; B06B 1/06; H10N 30/088

(52) U.S. Cl. CPC A61B 8/4494; A61B 8/12; B06B 1/0633; H10N 30/088; A61B 2562/12; B06B 2201/76

(57) ABSTRACT

An ultrasound transducer includes: a piezoelectric element layer including a piezoelectric element configured to transmit and receive ultrasound; a dematching layer that is layered over the piezoelectric element layer, the dematching layer being configured to reflect at least part of the ultrasound and including a first portion and a second portion that is higher in electric conductivity than the first portion, the second portion including a first surface positioned between the piezoelectric element layer and the first portion and a connection surface that is a surface different from the first surface, is thicker than the first surface, and is electrically connected to the first surface; and a wire electrically connected to the connection surface.

### CROSS-REFERENCE TO RELATED APPLICATION

This application is a continuation of International Application No. PCT/JP2022/038484, filed on Oct. 14, 2022, the entire contents of which are incorporated herein by reference.

### BACKGROUND

### 1. Technical Field

The present disclosure relates to an ultrasound transducer and a medical device.

### 2. Related Art

An ultrasound transducer having a piezoelectric element that transmits and receives ultrasound has been known conventionally (see, for example, Japanese Patent Application Laid-open No. 2013-077940). An ultrasound transducer in Patent Literature 1 has a dematching layer and a flexible printed circuit (FPC) layered over each other, the dematching layer being a layer that is on a back surface of a piezoelectric element and that reflects ultrasound, the FPC being for transmitting and receiving electric signals to and from the piezoelectric element.

### SUMMARY

In some embodiments, an ultrasound transducer includes: a piezoelectric element layer including a piezoelectric element configured to transmit and receive ultrasound; a dematching layer that is layered over the piezoelectric element layer, the dematching layer being configured to reflect at least part of the ultrasound and including a first portion and a second portion that is higher in electric conductivity than the first portion, the second portion including a first surface positioned between the piezoelectric element layer and the first portion and a connection surface that is a surface different from the first surface, is thicker than the first surface, and is electrically connected to the first surface; and a wire electrically connected to the connection surface.

In some embodiments, an ultrasound transducer includes: a piezoelectric element layer including a piezoelectric element configured to transmit and receive ultrasound; a dematching layer that is layered over the piezoelectric element layer, the dematching layer being configured to reflect at least part of the ultrasound, the dematching layer including a first portion and a second portion that is higher in electric conductivity than the first portion, the first portion including a recessed portion, the second portion including a protruded portion fitted in the recessed portion; and a wire electrically connected to a back surface of the protruded portion of the second portion.

In some embodiments, an ultrasound transducer includes: a piezoelectric element layer including a piezoelectric element configured to transmit and receive ultrasound; and a dematching layer that is layered over the piezoelectric element layer, the dematching layer being configured to reflect at least part of the ultrasound, the dematching layer including a first portion and a second portion that is higher in electric conductivity than the first portion, the first portion and the second portion being made of the same materials, a concentration of an electrically conductive material included in the second portion being higher than a concentration of the electrically conductive material included in the first portion.

In some embodiments, a medical device includes: the ultrasound transducer; and an insertion portion having a distal end provided with the ultrasound transducer, the insertion portion being configured to be inserted into a subject.

The above and other features, advantages and technical and industrial significance of this disclosure will be better understood by reading the following detailed description of presently preferred embodiments of the disclosure, when considered in connection with the accompanying drawings.

### BRIEF DESCRIPTION OF THE DRAWINGS

FIG. 1 is a schematic diagram illustrating an overall endoscope system;

FIG. 2 is a perspective view illustrating a distal end of an insertion portion;

FIG. 3 is a sectional diagram illustrating a configuration of an ultrasound transducer including ultrasound transducers according to a first embodiment;

FIG. 4 is a sectional diagram corresponding to a line A-A in FIG. 3;

FIG. 5 is a flowchart illustrating an outline of steps of a method of manufacturing the ultrasound transducer according to the first embodiment;

FIG. 6 is a diagram illustrating how components are prepared;

FIG. 7 is a diagram illustrating how the components are layered;

FIG. 8 is a diagram illustrating how a wire is connected;

FIG. 9 is a flowchart illustrating an outline of steps of a method of manufacturing an ultrasound transducer according to a second embodiment;

FIG. 10 is a diagram illustrating how components are prepared;

FIG. 11 is a diagram illustrating how the components are layered;

FIG. 12 is a sectional diagram of an ultrasound transducer according to a third embodiment;

FIG. 13 is a diagram illustrating how components are prepared;

FIG. 14 is a diagram illustrating how the components are layered;

FIG. 15 is a diagram illustrating how a wire is connected;

FIG. 16 is a flowchart illustrating an outline of steps of a method of manufacturing an ultrasound transducer according to a fourth embodiment;

FIG. 17 is a sectional diagram of an ultrasound transducer according to a fifth embodiment;

FIG. 18 is a diagram illustrating how components are prepared;

FIG. 19 is a diagram illustrating how the components are layered;

FIG. 20 is a diagram illustrating how a wire is connected;

FIG. 21 is a flowchart illustrating an outline of steps of a method of manufacturing an ultrasound transducer according to a sixth embodiment;

FIG. 22 is a diagram illustrating how components are prepared;

FIG. 23 is a diagram illustrating how the components are layered;

FIG. 24 is a sectional diagram of an ultrasound transducer according to a seventh embodiment;

FIG. 25 is a sectional diagram of an ultrasound transducer according to an eighth embodiment; and

FIG. 26 is a sectional diagram of an ultrasound transducer according to a ninth embodiment.

### DETAILED DESCRIPTION

Embodiments of an ultrasound transducer, a medical device, and a method of manufacturing the ultrasound transducer, according to the disclosure will be described hereinafter by reference to the drawings. The disclosure is not to be limited by these embodiments. The disclosure is applicable generally to ultrasound transducers, medical devices, and methods of manufacturing the ultrasound transducers.

Any elements that are the same or corresponding to each other are assigned with the same reference sign throughout the drawings, as appropriate. It also needs to be noted that the drawings are schematic, and relations between dimensions of each element therein and proportions between the elements therein may be different from the actual relations and proportions. The drawings may also include a portion that differs in its dimensional relations or proportions among the drawings.

### First Embodiment

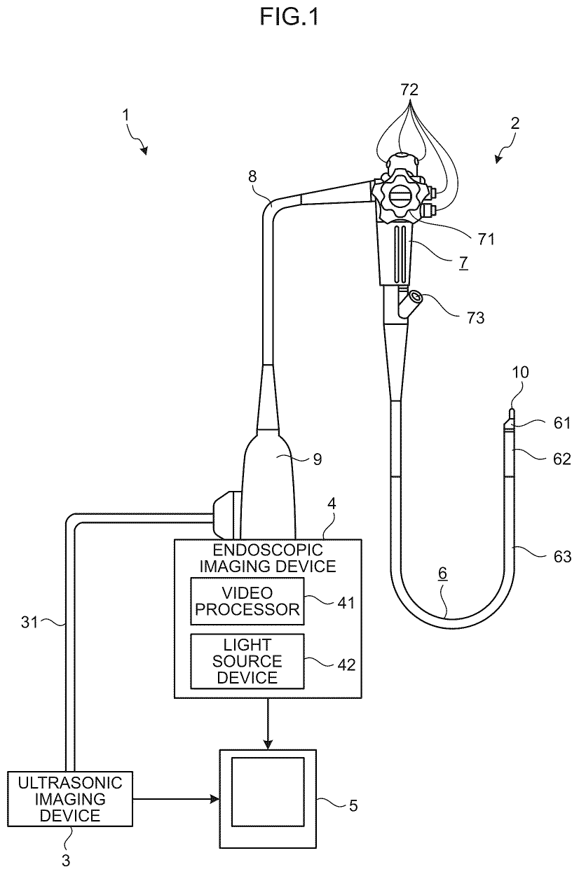

Schematic Configuration of Endoscope System FIG. 1 is a schematic diagram illustrating an overall endoscope system. An endoscope system 1 serving as a medical device is a system for ultrasound diagnosis and treatment of the interior of a subject, such as a human, using an ultrasound endoscope. This endoscope system 1 includes, as illustrated in FIG. 1, an ultrasound endoscope 2, an ultrasonic imaging device 3, an endoscopic imaging device 4, and a display device 5.

Part of the ultrasound endoscope 2 is able to be inserted into a subject, and the ultrasound endoscope 2 has: a function of transmitting ultrasound pulses (acoustic pulses) to a body wall inside the subject, receiving an ultrasound echo reflected in the subject, and outputting an echo signal; and a function of capturing an image of the interior of the subject and outputting an image signal. A detailed configuration of the ultrasound endoscope 2 will be described later.

The ultrasonic imaging device 3 is electrically connected to the ultrasound endoscope 2 via an ultrasound cable 31, and outputs a pulse signal to the ultrasound endoscope 2 and inputs an echo signal from the ultrasound endoscope 2, via the ultrasound cable 31. The ultrasonic imaging device 3 generates an ultrasound image by performing predetermined processing of the echo signal.

An endoscope connector 9 of the ultrasound endoscope 2 is detachably connected to the endoscopic imaging device 4. The endoscopic imaging device 4 includes, as illustrated in FIG. 1, a video processor 41 and a light source device 42.

The video processor 41 receives an image signal from the ultrasound endoscope 2 via the endoscope connector 9. The video processor 41 generates an endoscopic image by performing predetermined processing of the image signal.

The light source device 42 supplies illumination light for illuminating the interior of a subject, to the ultrasound endoscope 2 via the endoscope connector 9.

The display device 5 is formed using liquid crystal, organic electroluminescence (EL), a cathode ray tube (CRT), or a projector, and displays, for example, an ultrasound image generated by the ultrasonic imaging device 3 or an endoscopic image generated by the endoscopic imaging device 4.

Configuration of Ultrasound Endoscope A configuration of the ultrasound endoscope 2 will be described next. The ultrasound endoscope 2 includes, as illustrated in FIG. 1, an insertion portion 6, an operating portion 7, a universal cord 8, and the endoscope connector 9.

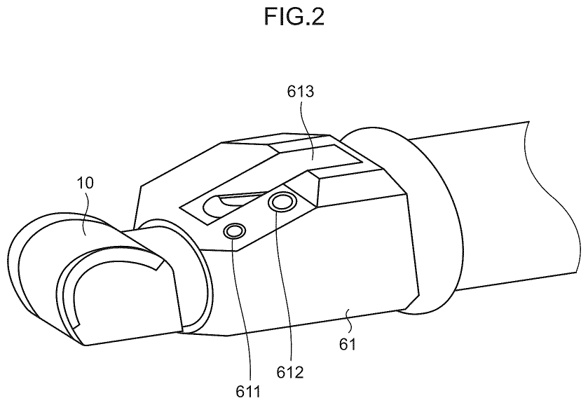

FIG. 2 is a perspective view illustrating a distal end of an insertion portion. In describing a configuration of the insertion portion 6 hereinafter, a distal end of the insertion portion 6 (the distal end in a direction in which the insertion portion 6 is inserted into a subject) will simply be referred to as “the distal end” and a proximal end of the insertion portion 6 (its end away from the distal end of the insertion portion 6) will simply be referred to as “the proximal end”.

The insertion portion 6 is a portion to be inserted into a subject. This insertion portion 6 includes, as illustrated in FIG. 1 or FIG. 2, an ultrasound probe 10 provided at the distal end; a rigid portion 61 connected to a proximal end of the ultrasound probe 10; a bending portion 62 that is connected to a proximal end of the rigid portion 61 and is bendable; and a flexible tube 63 (FIG. 1) that is connected to a proximal end of the bending portion 62 and has flexibility.

The insertion portion 6, the operating portion 7, the universal cord 8, and the endoscope connector 9 have: laid therethrough, a light guide that transmits illumination light supplied from the light source device 42, a transducer cable that transmits pulse signals and echo signals, and a signal cable that transmits image signals; and provided therein, a channel for circulating fluid.

The rigid portion 61 is a rigid part including a resin material, for example. As illustrated in FIG. 2, an illumination portion 611 that illuminates the interior of a subject with illumination light, an imager 612 that captures an image of the interior of the subject, and a treatment tool channel 613 that lets a treatment tool protrude from the distal end of the insertion portion 6 have been arranged at a distal end of the rigid portion 61.

The illumination portion 611 includes: a light guide that is arranged at a distal end of the illumination portion 611 and that transmits illumination light output by the light source device 42 to the distal end of the insertion portion 6; and an illumination lens that outputs the illumination light output from an output end of the light guide to the interior of a subject.

The imager 612 captures an image of the interior of a subject. The imager 612 has: an objective optical system that condenses light (a subject image) that has been emitted into the subject and reflected in the subject; and an imaging element that captures the subject image condensed by the objective optical system. An image signal captured by the imaging element is transmitted to the endoscopic imaging device 4 (the video processor 41) via the signal cable.

The treatment tool channel 613 is a channel that lets a treatment tool protrude outside, the treatment tool being, for example, a puncture needle, which has been inserted through the insertion portion 6.

The operating portion 7 is a portion that is connected to the proximal end of the insertion portion 6 and receives various kinds of operation from a medical doctor, for example. This operating portion 7 includes, as illustrated in FIG. 1, a bending knob 71 for bending operation of the bending portion 62, and plural operating parts 72 for performing various kinds of operation.

Furthermore, the operating portion 7 has, provided therein, a treatment tool insertion port 73 (FIG. 1) that communicates with the treatment tool channel 613 via a tube provided inside the bending portion 62 and the flexible tube 63, the treatment tool insertion port 73 being for insertion of a treatment tool through the tube.

The universal cord 8 is a cord extending from the operating portion 7 and having, arranged therein, a tube forming part of: the light guide; the transducer cable; the signal cable; and the channel.

The endoscope connector 9 is provided at an end portion of the universal cord 8. The ultrasound cable 31 is connected to the endoscope connector 9, and the endoscope connector 9 is connected to the video processor 41 and the light source device 42 by being plugged into the endoscopic imaging device 4.

### Configuration of Ultrasound Probe

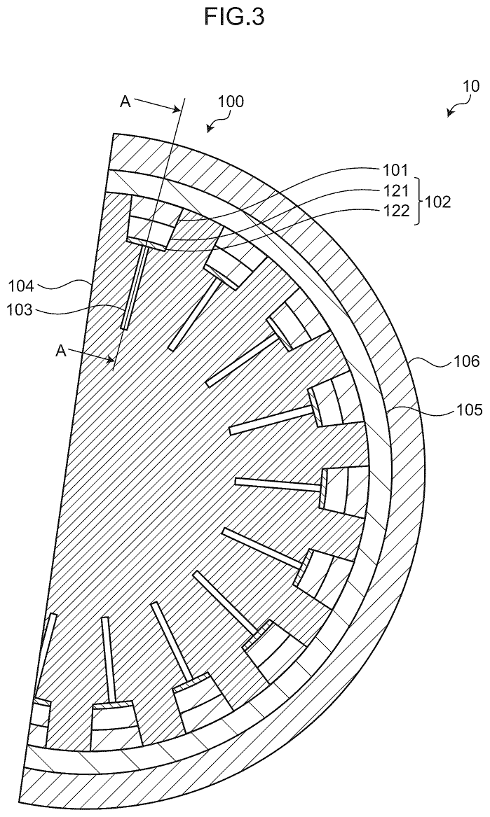

A configuration of the ultrasound probe 10 will be described next. FIG. 3 is a sectional diagram illustrating a configuration of an ultrasound transducer including ultrasound transducers according to a first embodiment. As illustrated in FIG. 3, the ultrasound probe 10 is a convex ultrasound transducer including plural ultrasound transducers 100 arranged in a circular arc shape, but may be a radial or linear ultrasound transducer.

### Configuration of Ultrasound Transducers

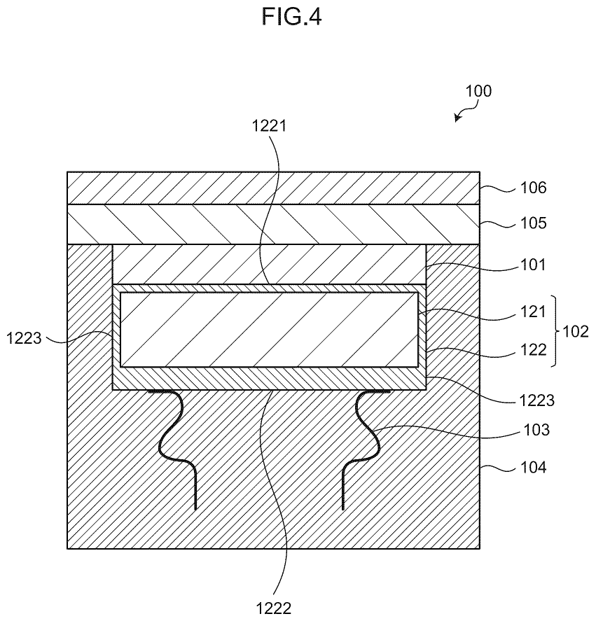

A configuration of the ultrasound transducers 100 will be described next. FIG. 4 is a sectional diagram corresponding to a line A-A in FIG. 3. As illustrated in FIG. 4, the ultrasound transducers 100 each include a piezoelectric element layer 101, a dematching layer 102, a wire 103, a backing material 104, a first acoustic matching layer 105, and a second acoustic matching layer 106.

The piezoelectric element layer 101 includes a piezoelectric element that transmits and receives ultrasound. The piezoelectric element layer 101 is formed of a long cuboid having its long sides extending from/to left to/from right in FIG. 4. The piezoelectric element layer 101 converts a pulse signal input via the wire 103 and the dematching layer 102 into ultrasound pulses and transmits the ultrasound pulses to a subject. Furthermore, the piezoelectric element layer 101 converts an ultrasound echo reflected by the subject into an electric echo signal representing the ultrasound echo by change in voltage and outputs the electric echo signal to the wire 103 via the dematching layer 102.

The piezoelectric element is formed using PMN-PT single crystal, PMN-PZT single crystal, PZN-PT single crystal, PIN-PZN-PT single crystal, or a relaxor-based material. PMN-PT single crystal is an abbreviation for a solid solution of lead magnesium niobate and lead titanate. PMN-PZT single crystal is an abbreviation for a solid solution of lead magnesium niobate and lead zirconate titanate. PZN-PT single crystal is an abbreviation for a solid solution of lead zinc niobate and lead titanate. PIN-PZN-PT single crystal is an abbreviation for a solid solution of lead indium niobate, lead zinc niobate, and lead titanate. The relaxor-based material is a general term for ternary system piezoelectric materials including lead zirconate titanate (PZT) and lead-based complex perovskite that is added as a relaxor material to increase the piezoelectric constant and dielectric constant. Lead-based complex perovskite is expressed as Pb(B1,B2)O₃ where B1 is magnesium, zinc, indium, or scandium, and B2 is niobium, tantalum, or tungsten. These materials have excellent piezoelectric effect. Therefore, even if downsizing is implemented, the value of electric impedance is able to be maintained low.

The dematching layer 102 is layered over the piezoelectric element layer 101 and reflects at least part of ultrasound. The dematching layer 102 has a first portion 121 and a second portion 122.

The first portion 121 is made of a material higher in acoustic impedance than the second portion 122. The first portion 121 is, for example, tungsten carbide high in acoustic impedance, but may include at least one of tungsten (W), cobalt (Co), nickel (Ni), titanium (Ti), chromium (Cr), molybdenum (Mo), tantalum (Ta), or carbon (C).

The second portion 122 is higher in electric conductivity than the first portion 121. The second portion 122 is, for example, gold (Au), but may include at least one of silver (Ag), copper (Cu), cobalt (Co), nickel (Ni), titanium (Ti), chromium (Cr), molybdenum (Mo), tantalum (Ta), or carbon (C).

The second portion 122 has a first surface 1221 positioned between the piezoelectric element layer 101 and the first portion 121, a second surface 1222 positioned to be across the first portion 121 from the first surface 1221, and a third surface 1223 connected to the first surface 1221 and the second surface 1222. The second surface 1222 has a thickness larger than a thickness of the first surface 1221 and a thickness of the third surface 1223. Furthermore, the thickness of the second surface 1222 is preferably ½ or less, and more preferably ⅓ or less, of a thickness of the dematching layer 102. Not making the thickness of the second surface 1222 too large enables prevention of reduction in the ultrasound reflecting effect of the dematching layer 102. The thickness of the second surface 1222 is a dimension of the second surface 1222, the dimension being along a direction perpendicular to the second surface 1222. Similarly, the thickness of the first surface 1221 and the thickness of the third surface 1223 are dimensions of the first surface 1221 and the third surface 1223, the dimensions being along directions perpendicular to the first surface 1221 and the third surface 1223. The thickness of the dematching layer 102 is a dimension of the dematching layer 102, the dimension being along a direction that the dematching layer 102 and the piezoelectric element layer 101 are layered in.

One end of the wire 103 has been electrically connected to the second surface 1222 of the second portion 122 with solder but may be electrically connected by ultrasonic welding or brazing, for example. The other end of the wire 103 is electrically connected to the ultrasonic imaging device 3 via the universal cord 8 and the ultrasound cable 31 but illustration of this connection has been omitted in FIG. 4. The wire 103 transmits a pulse signal output from the ultrasonic imaging device 3 to the piezoelectric element layer 101 and transmits an echo signal output by the piezoelectric element layer 101 to the ultrasonic imaging device 3. The wire 103 is made of a material different from that of the second portion 122.

The backing material 104 surrounds the wire 103 and absorbs or attenuates unnecessary ultrasound generated by operation of the piezoelectric element layer 101. The backing material 104 is formed using a material having high absorptivity or a high attenuation rate, for example, epoxy resin having a filler, such as alumina or zirconia, dispersed therein, or rubber having the filler dispersed therein. The backing material 104 may be not provided and whether or not the backing material 104 is to be provided depends on properties of the piezoelectric element layer 101 and a target to be observed.

With respect to the piezoelectric element layers 101, the first acoustic matching layer 105 and the second acoustic matching layer 106 are positioned in directions (upward in FIG. 4) that the piezoelectric element layers 101 transmit ultrasound. Furthermore, as illustrated in FIG. 3, the first acoustic matching layer 105 and the second acoustic matching layer 106 are continuous along a direction that the piezoelectric element layers 101 are arranged in and the first acoustic matching layer 105 and the second acoustic matching layer 106 thereby hold the piezoelectric element layers 101 in a circular arc shape. The first acoustic matching layer 105 and the second acoustic matching layer 106 cause matching between acoustic impedances of the piezoelectric element layers 101 and a target to be observed to achieve efficient transmission of sound (ultrasound) between the piezoelectric element layers 101 and the target to be observed. The first acoustic matching layer 105 and the second acoustic matching layer 106 are made of materials different from each other. This first embodiment is described as having two acoustic matching layers (the first acoustic matching layer 105 and the second acoustic matching layer 106), but acoustic matching layers may be not provided or the number of acoustic matching layers provided may be one or three or more. Whether to provide any acoustic matching layer and how many acoustic matching layers are to be provided depend on the properties of the piezoelectric element layers 101 and the target to be observed.

### Method of Manufacturing Ultrasound Transducer

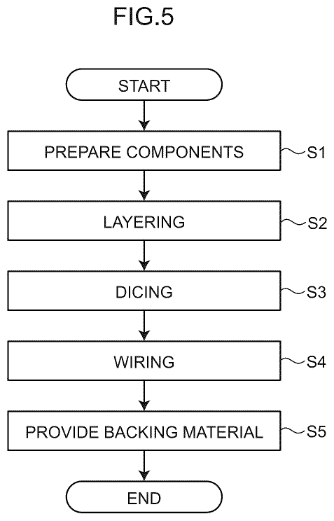

A method of manufacturing an ultrasound transducer will be described next. FIG. 5 is a flowchart illustrating an outline of steps of a method of manufacturing the ultrasound transducer according to the first embodiment.

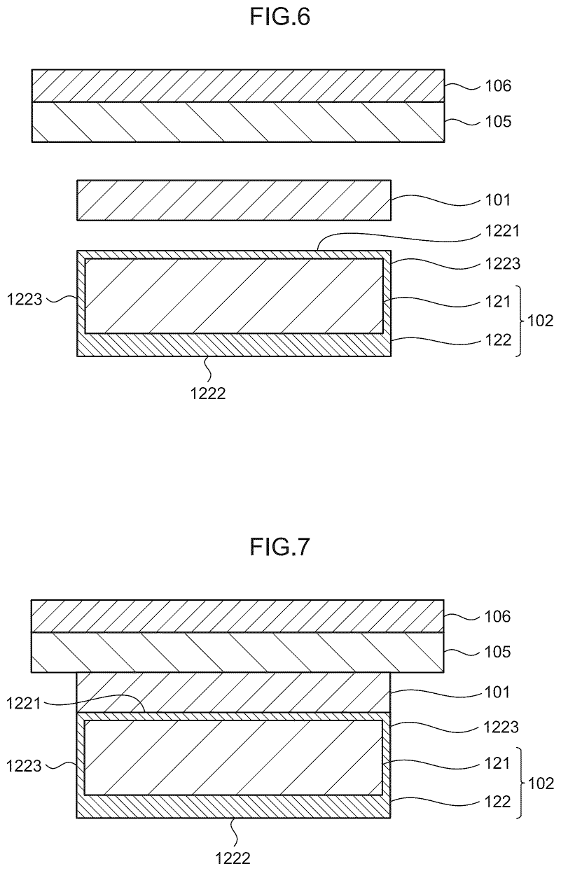

As illustrated in FIG. 5, components used for manufacturing the ultrasound transducer 100 are prepared (Step S1). FIG. 6 is a diagram illustrating how the components are prepared. As illustrated in FIG. 6, the piezoelectric element layer 101, the dematching layer 102, the backing material 104, the first acoustic matching layer 105, and the second acoustic matching layer 106 are prepared. The thickness of the second surface 1222 of the second portion 122 has been made larger than the thickness of the first surface 1221 and the thickness of the third surface 1223. This is able to be implemented by performing, for example, plating or sputtering, upon forming the second portion 122 on surfaces of the first portion 121, so that the thickness of the second surface 1222 becomes larger than the thickness of the first surface 1221 and the thickness of the third surface 1223.

Subsequently, the piezoelectric element layer 101, the dematching layer 102, the backing material 104, the first acoustic matching layer 105, and the second acoustic matching layer 106 are layered over one another with an adhesive, for example (Step S2). FIG. 7 is a diagram illustrating how the components are layered. As illustrated in FIG. 7, a layered product having the dematching layer 102 to the second acoustic matching layer 106 layered over one another is formed.

Thereafter, dicing to cut the layered product formed at Step S2 with a dicing saw is performed (Step S3). The dematching layer 102 and the piezoelectric element layer 101 are thereby cut and the plate-shaped piezoelectric element layer 101 is thus formed into a cuboid.

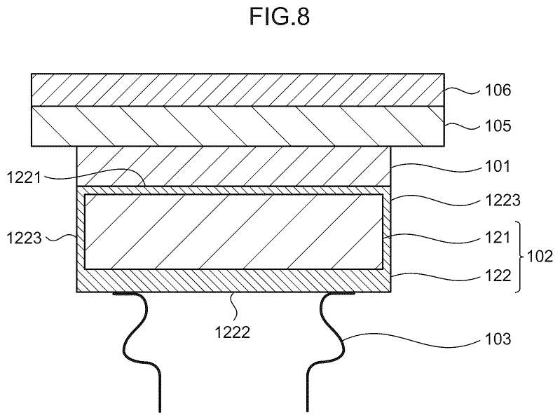

Wiring is attached to the ultrasound transducer 100 (Step S4). FIG. 8 is a diagram illustrating how a wire is connected. As illustrated in FIG. 8, the wire 103 is electrically connected to the second surface 1222 of the second portion 122 with solder.

Lastly, in a state where the first acoustic matching layer 105 and the second acoustic matching layer 106 have been bent in the circular arc shape illustrated in FIG. 3, a region on one side of the piezoelectric element layer 101 is filled with the backing material 104, the one side being where the dematching layer 102 has been layered over the piezoelectric element layer 101 (Step S5). As a result, the ultrasound probe 10 having the plural ultrasound transducers 100 is manufactured.

According to the first embodiment described above, at Step S4, the second surface 1222 of the second portion 122 has been made thick when the wire 103 is electrically connected to the second surface 1222 with solder. As a result, the second portion 122 is able to be prevented from melting and being absorbed by the solder (taken in by the solder) and connection failures are thus able to be prevented.

Furthermore, the ultrasound transducer 100 according to the first embodiment does not have an FPC and noise due to reflection by an FPC is thus prevented from being generated.

Furthermore, according to the first embodiment, the dematching layer 102 and the piezoelectric element layer 101 are cut by dicing, and bending is performed on the basis of the first acoustic matching layer 105 and the second acoustic matching layer 106 positioned outside the piezoelectric elements. Pitches between the piezoelectric elements are thereby able to be maintained upon the bending. By contrast, if the piezoelectric element layer 101, the first acoustic matching layer 105, and the second acoustic matching layer 106 were to be cut by dicing and bending is performed on the basis of, for example, an FPC positioned inside the piezoelectric elements, the pitches between the piezoelectric elements would be increased upon the bending and the pitches would thus be unable to be maintained.

### Second Embodiment

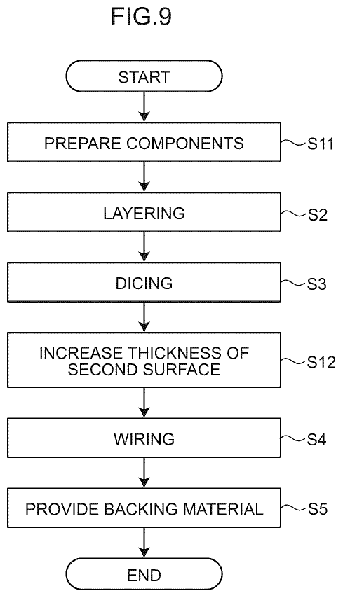

A configuration of an ultrasound transducer 100 according to a second embodiment is similar to that of the first embodiment and description thereof will thus be omitted. FIG. 9 is a flowchart illustrating an outline of steps of a method of manufacturing an ultrasound transducer according to the second embodiment.

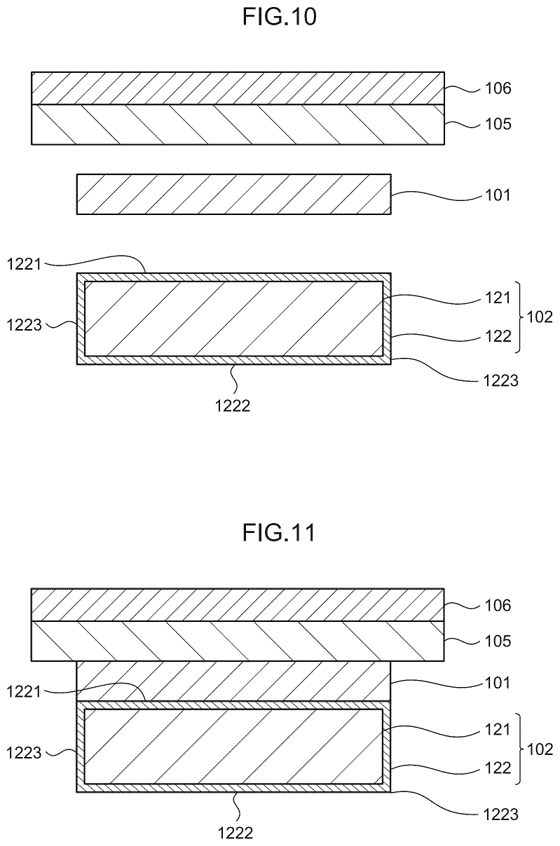

As illustrated in FIG. 9, components used for manufacturing the ultrasound transducer 100 are prepared (Step S11). FIG. 10 is a diagram illustrating how the components are prepared. As illustrated in FIG. 10, a piezoelectric element layer 101, a dematching layer 102, a backing material 104, a first acoustic matching layer 105, and a second acoustic matching layer 106 are prepared. A first surface 1221, a second surface 1222, and a third surface 1223 of a second portion 122 prepared have the same thickness. The thickness of each of these surfaces is a dimension of the surface, the dimension being along a direction perpendicular to the surface.

Subsequently, the piezoelectric element layer 101, the dematching layer 102, the backing material 104, the first acoustic matching layer 105, and the second acoustic matching layer 106 are layered over one another with an adhesive, for example (Step S2). FIG. 11 is a diagram illustrating how the components are layered. As illustrated in FIG. 11, a layered product having the dematching layer 102 to the second acoustic matching layer 106 layered over one another is formed.

Thereafter, dicing to cut the layered product formed at Step S2 with a dicing saw is performed (Step S3). The dematching layer 102 and the piezoelectric element layer 101 are thereby cut and the plate-shaped piezoelectric element layer 101 is thus formed into a cuboid.

The thickness of the second surface 1222 of the dematching layer 102 is then increased (Step S12). This may be implemented by, for example, plating or sputtering performed on a back surface of the layered product. A layered product similar to that in FIG. 7 is thereby formed. Steps after Step S12 may be similar to those of the first embodiment and description thereof will thus be omitted.

Like in the second embodiment described above, the thickness of the second surface 1222 of the second portion 122 may be increased after the layers have been layered over one another. In this case also, similarly to the first embodiment, the second portion 122 is able to be prevented from melting and being absorbed by solder (taken in by the solder) and connection failures are thus able to be prevented.

### Third Embodiment

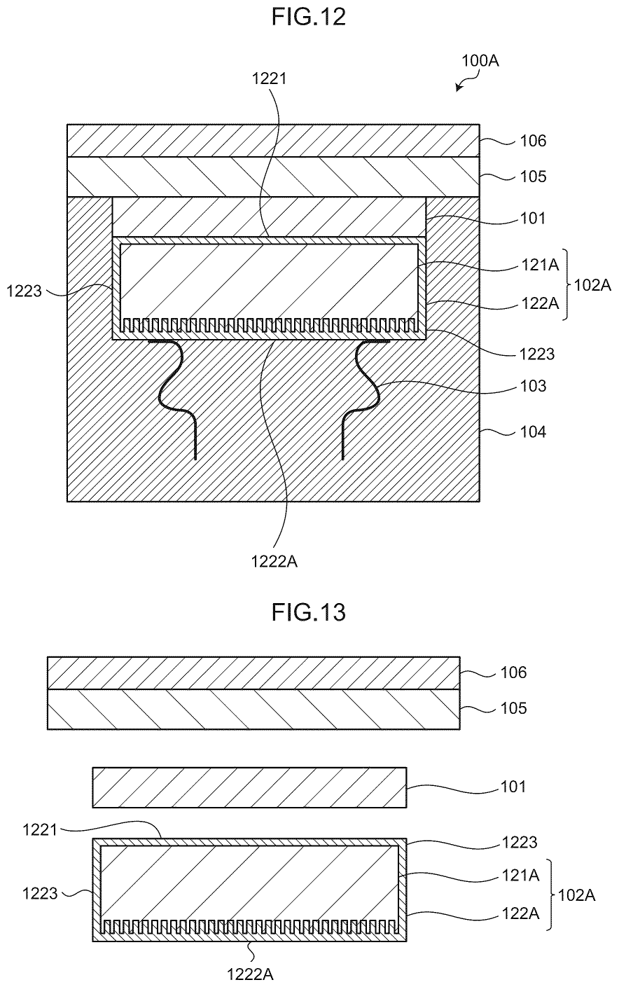

FIG. 12 is a sectional view of an ultrasound transducer according to a third embodiment. As illustrated in FIG. 12, a dematching layer 102A of an ultrasound transducer 100A according to the third embodiment has a surface with protrusions and recesses, the surface having been formed between a first portion 121A and a second portion 122A. A wire 103 is electrically connected to a second surface 1222A of the second portion 122A, the second surface 1222A being where the surface with protrusions and recesses has been formed.

A method of manufacturing the ultrasound transducer 100A will be described next. A method of manufacturing an ultrasound transducer according to the third embodiment has steps similar to those in FIG. 5.

As illustrated in FIG. 5, components used for manufacturing the ultrasound transducer 100A are prepared (Step S1). FIG. 13 is a diagram illustrating how the components are prepared. As illustrated in FIG. 13, a piezoelectric element layer 101, the dematching layer 102A, a backing material 104, a first acoustic matching layer 105, and a second acoustic matching layer 106 are prepared. When these components are prepared, the surface with protrusions and recesses has been formed between the first portion 121A and the second portion 122A of the dematching layer 102A. This can be implemented by plating performed to fill recessed portions when the second portion 122A is formed by plating on a surface of the first portion 121A, the surface being where a surface with recesses and protrusions has been formed.

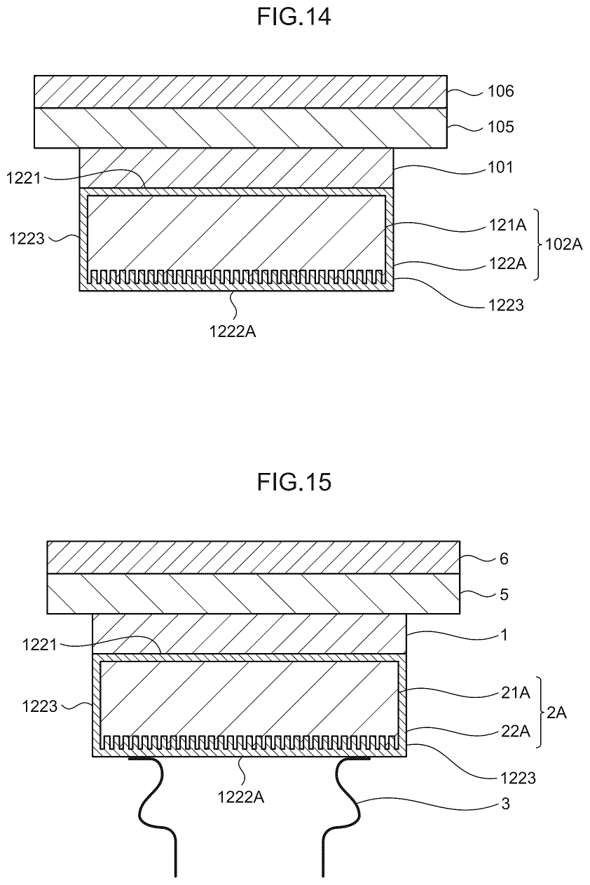

Subsequently, the piezoelectric element layer 101, the dematching layer 102A, the backing material 104, the first acoustic matching layer 105, and the second acoustic matching layer 106 are layered over one another with an adhesive, for example (Step S2). FIG. 14 is a diagram illustrating how the components are layered. As illustrated in FIG. 14, a layered product having the dematching layer 102A to the second acoustic matching layer 106 layered over one another is formed.

Thereafter, dicing to cut the layered product formed at Step S2 with a dicing saw is performed (Step S3). The dematching layer 102A and the piezoelectric element layer 101 are thereby cut and the plate-shaped piezoelectric element layer 101 is formed into a cuboid.

Wiring is then attached to the ultrasound transducer 100A (Step S4). FIG. 15 is a diagram illustrating how a wire is connected. As illustrated in FIG. 15, the wire 103 is electrically connected to the second surface 1222A of the second portion 122A with solder.

Lastly, in a state where the first acoustic matching layer 105 and the second acoustic matching layer 106 have been bent in the circular arc shape illustrated in FIG. 3, a region on one side of the piezoelectric element layer 101 is filled with the backing material 104, the one side being where the dematching layer 102A is layered over the piezoelectric element layer 101 (Step S5). As a result, an ultrasound probe having a plurality of the ultrasound transducers 100A is manufactured.

According to the third embodiment described above, at Step S4, the surface with the protrusions and recesses has been formed between the first portion 121A and the second surface 1222A when the wire 103 is electrically connected to the second surface 1222A of the second portion 122A with solder. As a result, the second portion 122A is able to be prevented from melting and being absorbed by the solder (taken in by the solder) and connection failures are thus able to be prevented.

### Fourth Embodiment

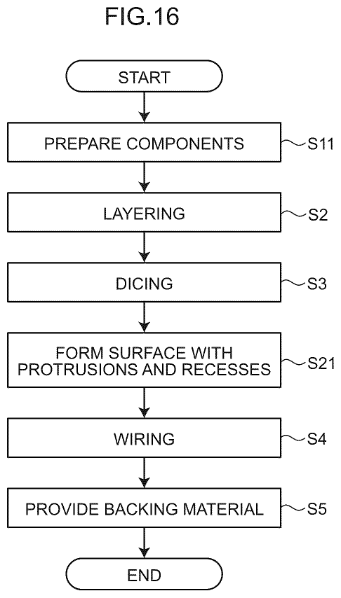

A configuration of an ultrasound transducer 100A according to a fourth embodiment is similar to that of the third embodiment and description thereof will thus be omitted. FIG. 16 is a flowchart illustrating an outline of steps of a method of manufacturing an ultrasound transducer according to the fourth embodiment.

As illustrated in FIG. 16, processes similar to those in FIG. 9 are performed at steps up to and including Step S3, and a dematching layer 102 and a piezoelectric element layer 101 are thereby cut and a layered product having the dematching layer 102 to a second acoustic matching layer 106 layered over one another is thus formed, as illustrated in FIG. 11. In this layered product, a first surface 1221, a second surface 1222, and a third surface 1223 of a second portion 122 have the same thickness. The thickness of each of these surfaces is a dimension of the surface, the dimension being along a direction perpendicular to the surface.

Subsequently, a surface with protrusions and recesses is formed between a first portion 121A and a second portion 122A of the dematching layer 102A (Step S21). This is able to be implemented by: formation of the surface with protrusions and recesses on a back surface of the layered product by, for example, etching, laser irradiation, or sand blasting; and performing plating to fill recessed portions, or sticking a part high in electric conductivity onto the back surface. A layered product similar to that in FIG. 14 is thereby formed. Steps after Step S21 may be similar to those of the third embodiment and description thereof will thus be omitted.

Like in the fourth embodiment described above, the surface with protrusions and recesses may be formed between the first portion 121A and the second portion 122A of the dematching layer 102A after the layers have been layered over one another. In this case also, similarly to the third embodiment, the second portion 122A is able to be prevented from melting and being absorbed by solder (taken in by the solder) and connection failures are thus able to be prevented.

### Fifth Embodiment

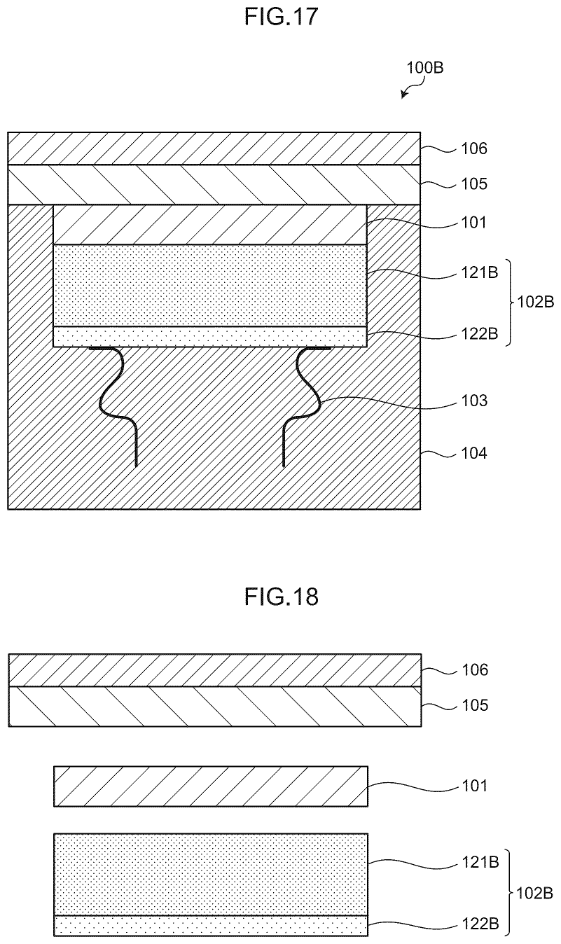

FIG. 17 is a sectional view of an ultrasound transducer according to a fifth embodiment. As illustrated in FIG. 17, a dematching layer 102B of an ultrasound transducer 100B according to the fifth embodiment includes a first portion 121B, and a second portion 122B layered on a surface of the first portion 121B, the surface of the first portion 121B being a surface away from the piezoelectric element layer 101.

The first portion 121B and the second portion 122B are made from the same materials, but a concentration of an electrically conductive material in the second portion 122B is higher than that in the first portion 121B, and the second portion 122B is thus higher in electric conductivity than the first portion 121B. Specifically, the first portion 121B and the second portion 122B are formed by, for example, addition of a filler having electric conductivity in tungsten carbide, but a concentration of the filler in the second portion 122B is higher than that in the first portion 121B. Furthermore, the second portion 122B has been formed thick to enable the second portion 122B to be prevented from being absorbed by solder when a wire 103 is connected to the second portion 122B. The second portion 122B has a thickness that is preferably ½ or less and more preferably ⅓ or less, of a thickness of the dematching layer 102B. The thickness of the dematching layer 102B and the thickness of the second portion 122B are their respective dimensions along a direction that the first portion 121B and the second portion 122B are arranged in. In other words, the thickness of the dematching layer 102B and the thickness of the second portion 122B are their dimensions along a direction perpendicular to the surface where soldering is performed.

To make the concentration of the electrically conductive material in the second portion 122B higher than that in the first portion 121B, the first portion 121B and the second portion 122B may be formed by making the concentration of a binder in part of tungsten carbide higher, for example. That is, the part of tungsten carbide may serve as the second portion 122B, and part comparatively lower in concentration of the binder may serve as the first portion 121B. Furthermore, the first portion 121B and the second portion 122B may be formed separately and connected to each other. That is, two materials containing tungsten carbide and having binder or filler concentrations different from each other may be formed and connected to each other.

A method of manufacturing an ultrasound transducer will be described next. A method of manufacturing the ultrasound transducer according to the fifth embodiment has steps similar to those in FIG. 5.

As illustrated in FIG. 5, components used for manufacturing the ultrasound transducer 100B are prepared (Step S1). FIG. 18 is a diagram illustrating how the components are prepared. As illustrated in FIG. 18, the piezoelectric element layer 101, the dematching layer 102B, a backing material 104, a first acoustic matching layer 105, and a second acoustic matching layer 106 are prepared. The dematching layer 102B has the first portion 121B, and the second portion 122B higher in electric conductivity than the first portion 121B.

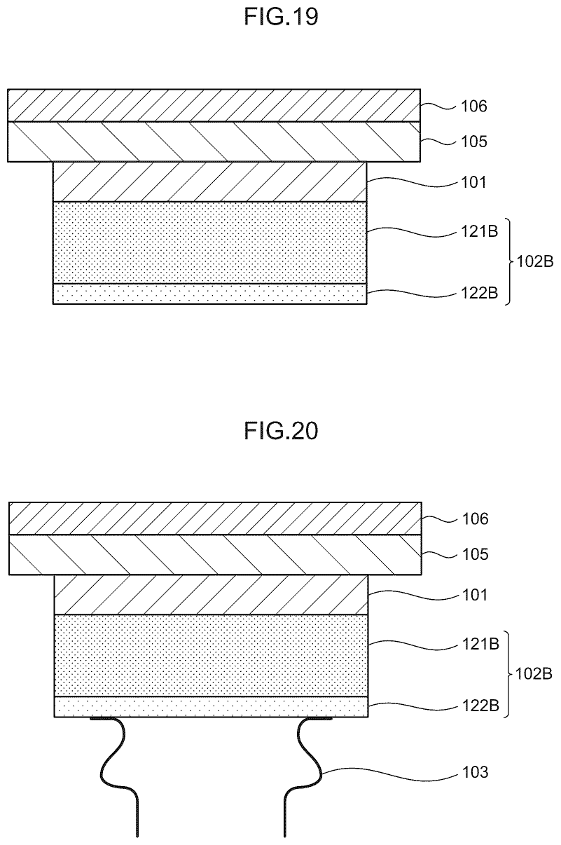

Subsequently, the piezoelectric element layer 101, the dematching layer 102B, the backing material 104, the first acoustic matching layer 105, and the second acoustic matching layer 106 are layered over one another with an adhesive, for example (Step S2). FIG. 19 is a diagram illustrating how the components are layered. As illustrated in FIG. 19, a layered product having the dematching layer 102B to the second acoustic matching layer 106 layered over one another is formed.

Thereafter, dicing to cut the layered product formed at Step S2 with a dicing saw is performed (Step S3). The dematching layer 102B and the piezoelectric element layer 101 are thereby cut and the plate-shaped piezoelectric element layer 101 is formed into a cuboid.

Wiring is then attached to the ultrasound transducer 100B (Step S4). FIG. 20 is a diagram illustrating how a wire is connected. As illustrated in FIG. 20, the wire 103 is electrically connected to the second portion 122B with solder.

Lastly, in a state where the first acoustic matching layer 105 and the second acoustic matching layer 106 have been bent in the circular arc shape illustrated in FIG. 3, a region on one side of the piezoelectric element layer 101 is filled with the backing material 104, the one side being where the dematching layer 102B is layered over the piezoelectric element layer 101 (Step S5). As a result, an ultrasound probe having a plurality of the ultrasound transducers 100B is manufactured.

According to the fifth embodiment described above, at Step S4, the second portion 122B has been made thick when the wire 103 is electrically connected to the second portion 122B with solder. As a result, the second portion 122B is able to be prevented from melting and being absorbed by the solder (taken in by the solder) and connection failures are thus able to be prevented.

### Sixth Embodiment

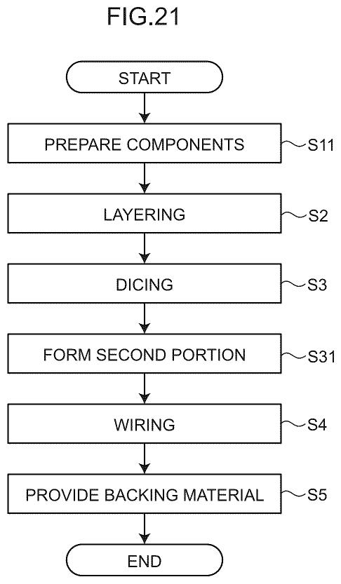

A configuration of an ultrasound transducer 100B according to a sixth embodiment is similar to that of the fifth embodiment and description thereof will thus be omitted. FIG. 21 is a flowchart illustrating an outline of steps of a method of manufacturing an ultrasound transducer according to the sixth embodiment.

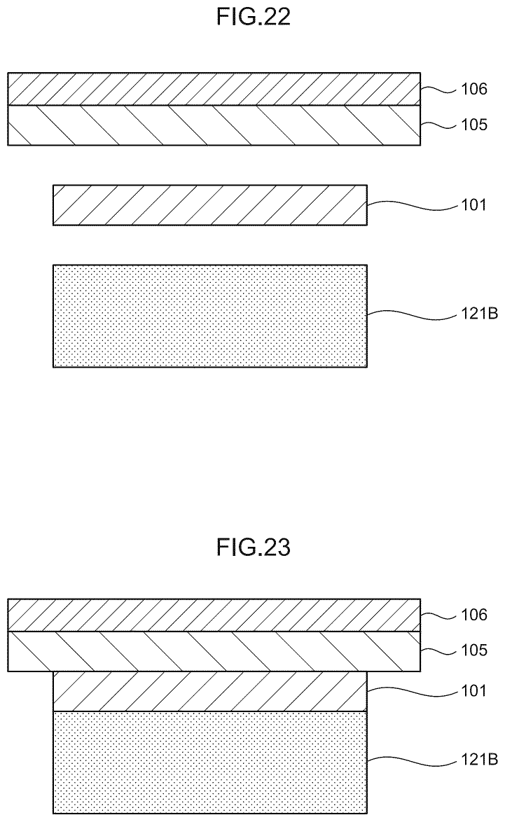

As illustrated in FIG. 21, components used for manufacturing the ultrasound transducer 100B are prepared (Step S11). FIG. 22 is a diagram illustrating how the components are prepared. As illustrated in FIG. 22, a piezoelectric element layer 101, a first portion 121B, a backing material 104, a first acoustic matching layer 105, and a second acoustic matching layer 106 are prepared.

Subsequently, the piezoelectric element layer 101, the first portion 121B, the backing material 104, the first acoustic matching layer 105, and the second acoustic matching layer 106 are layered over one another with an adhesive, for example (Step S2). FIG. 23 is a diagram illustrating how the components are layered. As illustrated in FIG. 23, a layered product having the first portion 121B to the second acoustic matching layer 106 layered over one another is formed.

Thereafter, dicing to cut the layered product formed at Step S2 with a dicing saw is performed (Step S3). The first portion 121B and the piezoelectric element layer 101 are thereby cut and the plate-shaped piezoelectric element layer 101 is formed into a cuboid.

The second portion 122B of the dematching layer 102B is then formed (Step S31). This is able to be implemented by performing plating, sputtering, or impregnation on the back surface of the layered product, or by sticking a part high in electric conductivity onto the back surface. A formed product similar to that in FIG. 7 is thereby formed, and steps after Step S31 may be similar to those of the first embodiment and description thereof will thus be omitted.

Like in the sixth embodiment described above, the second portion 122B may be formed after the layers have been layered over one another. In this case also, the second portion 122B is able to be prevented from melting and being absorbed by solder (taken in by the solder) and connection failures are thus able to be prevented.

### Seventh Embodiment

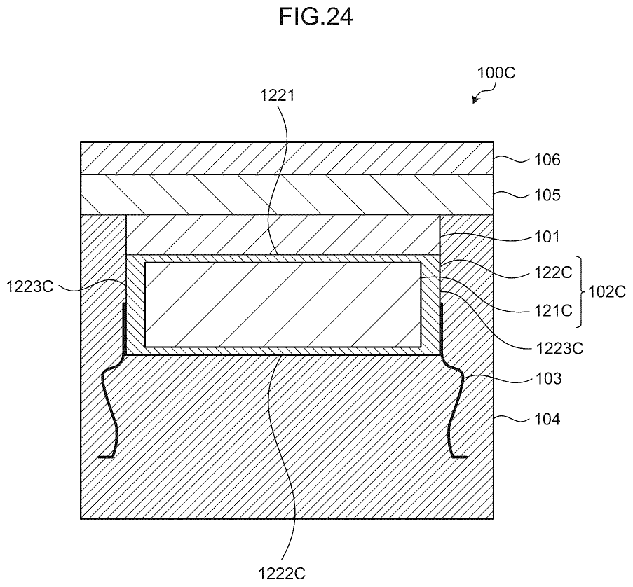

FIG. 24 is a sectional view of an ultrasound transducer according to a seventh embodiment. As illustrated in FIG. 24, a dematching layer 102C of an ultrasound transducer 100C according to the seventh embodiment has a first portion 121C and a second portion 122C. A third surface 1223C of the second portion 122C has a thickness larger than a thickness of a first surface 1221 and a thickness of a second surface 1222C. A wire 103 has been electrically connected to the third surface 1223C. The thickness of each of these surfaces is a dimension of the surface, the dimension being along a direction perpendicular to the surface.

According to the seventh embodiment described above, the third surface 1223C of the second portion 122 has been made thick when the wire 103 is electrically connected to the third surface 1223C with solder. As a result, the second portion 122C is able to be prevented from melting and being absorbed by the solder (taken in by the solder) and connection failures are thus able to be prevented.

### Eighth Embodiment

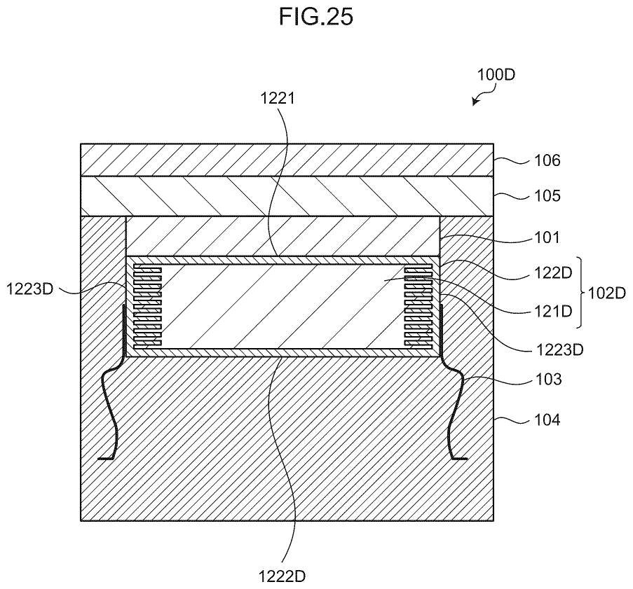

FIG. 25 is a sectional view of an ultrasound transducer according to an eighth embodiment. As illustrated in FIG. 25, a dematching layer 102D of an ultrasound transducer 100D according to the eighth embodiment has a surface with protrusions and recesses, the surface having been formed between a first portion 121D and a second portion 122D. A wire 103 is electrically connected to a third surface 1223D of the second portion 122D, the third surface 1223D being where the surface with protrusions and recesses has been formed. A second surface 1222D has the same thickness as a first surface 1221. The thickness of each of these surfaces is a dimension of the surface, the dimension being along a direction perpendicular to the surface.

According to the eighth embodiment described above, the surface with the protrusions and recesses has been formed between the first portion 121D and the third surface 1223D when the wire 103 is electrically connected to the third surface 1223D of the second portion 122D with solder. As a result, the second portion 122D is able to be prevented from melting and being absorbed by the solder (taken in by the solder) and connection failures are thus able to be prevented.

### Ninth Embodiment

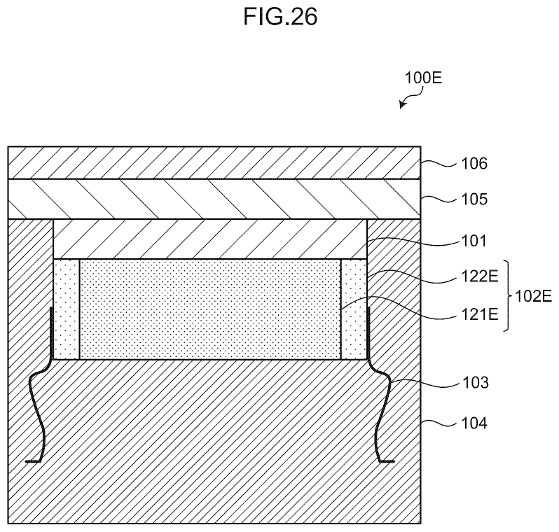

FIG. 26 is a sectional view of an ultrasound transducer according to a ninth embodiment. As illustrated in FIG. 26, a dematching layer 102E of an ultrasound transducer 100E according to the ninth embodiment has a first portion 121E, and a second portion 122E formed on a lateral surface of the first portion 121E.

The first portion 121E and the second portion 122E are made from the same materials, but a concentration of an electrically conductive material in the second portion 122E is higher than that in the first portion 121E and the second portion 122E is thus higher in electric conductivity than the first portion 121E. Furthermore, the second portion 122E has been formed thick to enable the second portion 122E to be prevented from being absorbed by solder when a wire 103 is connected to the second portion 122E. The thickness of the second portion 122E is preferably ½ or less and more preferably ⅓ or less, of a thickness of the dematching layer 102E. The thickness of the dematching layer 102E and the thickness of the second portion 122E are their respective dimensions along a direction that the first portion 121E and the second portion 122E are arranged in. In other words, the thickness of the dematching layer 102E and the thickness of the second portion 122E are their dimensions along a direction perpendicular to the surface where soldering is performed.

According to the ninth embodiment described above, the second portion 122E has been made thick when the wire 103 is electrically connected to the second portion 122E with solder. As a result, the second portion 122E is able to be prevented from melting and being absorbed by the solder (taken in by the solder) and connection failures are thus able to be prevented.

Ultrasound endoscopes have each been described thus far as a medical device, but the medical device may be an ultrasound catheter.

The disclosure enables implementation of an ultrasound transducer, a medical device, and a method of manufacturing the ultrasound transducer that enable noise to be reduced and connection failures to be prevented.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An ultrasound transducer, comprising:
a piezoelectric element layer including a piezoelectric element configured to transmit and receive ultrasound;
a dematching layer that is layered over the piezoelectric element layer, the dematching layer being configured to reflect at least part of the ultrasound and including a first portion and a second portion that is higher in electric conductivity than the first portion, the second portion including a first surface positioned between the piezoelectric element layer and the first portion and a connection surface that is a surface different from the first surface, is thicker than the first surface, and is electrically connected to the first surface; and
a wire electrically connected to the connection surface.

2. The ultrasound transducer according to claim 1, wherein the wire is made of a material different from a material of the second portion.

3. The ultrasound transducer according to claim 1, wherein the wire is electrically connected to the second portion with solder.

4. The ultrasound transducer according to claim 1, further comprising a backing material that surrounds the wire, the backing material being configured to absorb or attenuate the ultrasound.

5. The ultrasound transducer according to claim 1, wherein the first portion is made of a material higher in acoustic impedance than the second portion.

6. The ultrasound transducer according to claim 1, wherein the connection surface is a second surface positioned across the first portion from the first surface.

7. The ultrasound transducer according to claim 6, wherein the second surface has a thickness that is ⅓ or less of a thickness of the dematching layer.

8. The ultrasound transducer according to claim 1, wherein the connection surface is a third surface that connects the first surface and a second surface positioned across the first portion from the first surface.

9. The ultrasound transducer according to claim 1, wherein the first portion includes at least one of tungsten (W), cobalt (Co), nickel (Ni), titanium (Ti), chromium (Cr), molybdenum (Mo), tantalum (Ta), or carbon (C).

10. The ultrasound transducer according to claim 1, wherein the second portion includes at least one of gold (Au), silver (Ag), copper (Cu), cobalt (Co), nickel (Ni), titanium (Ti), chromium (Cr), molybdenum (Mo), tantalum (Ta), or carbon (C).

11. An ultrasound transducer, comprising:
a piezoelectric element layer including a piezoelectric element configured to transmit and receive ultrasound;
a dematching layer that is layered over the piezoelectric element layer, the dematching layer being configured to reflect at least part of the ultrasound, the dematching layer including a first portion and a second portion that is higher in electric conductivity than the first portion, the first portion including a recessed portion, the second portion including a protruded portion fitted in the recessed portion; and
a wire electrically connected to a back surface of the protruded portion of the second portion.

12. The ultrasound transducer according to claim 11, wherein the second portion including a first surface positioned between the piezoelectric element layer and the first portion and a second surface positioned across the first portion from the first surface, and the second surface has a thickness that is ⅓ or less of a thickness of the dematching layer.

13. An ultrasound transducer, comprising:
a piezoelectric element layer including a piezoelectric element configured to transmit and receive ultrasound; and
a dematching layer that is layered over the piezoelectric element layer, the dematching layer being configured to reflect at least part of the ultrasound, the dematching layer including a first portion and a second portion that is higher in electric conductivity than the first portion, the first portion and the second portion being made of the same materials, a concentration of an electrically conductive material included in the second portion being higher than a concentration of the electrically conductive material included in the first portion.

14. A medical device, comprising:
the ultrasound transducer according to claim 1; and
an insertion portion having a distal end provided with the ultrasound transducer, the insertion portion being configured to be inserted into a subject.

15. A medical device, comprising:
the ultrasound transducer according to claim 11; and
an insertion portion having a distal end provided with the ultrasound transducer, the insertion portion being configured to be inserted into a subject.

16. A medical device, comprising:
the ultrasound transducer according to claim 13; and
an insertion portion having a distal end provided with the ultrasound transducer, the insertion portion being configured to be inserted into a subject.

17. The medical device according to claim 14, further comprising an imager configured to capture an image of an interior of the subject.

18. The medical device according to claim 15, further comprising an imager configured to capture an image of an interior of the subject.

19. The medical device according to claim 16, further comprising an imager configured to capture an image of an interior of the subject.
